(19) United States
(12) Patent Application Publication
(10) Pub. No.: US 2022/0175263 A1
(43) Pub. Date: Jun. 9, 2022

(54) SYSTEMS, DEVICES AND METHODS FOR DETERMINING LARYNGOPHARYNGEAL PRESSURE AND/OR LOWER OESOPHAGEAL SPHINCTER PRESSURE

(71) Applicant: VENTORA MEDICAL PTY LTD, Docklands (AU)

(72) Inventors: Seetal Erramilli, Docklands (AU); Edward Charles Wilhelm Buijs, Docklands (AU); Lorinda Kathleen Hartley, Docklands (AU); Amy Yu, Docklands (AU); Alan Haszard, Docklands (AU); Christiane Theda, Docklands (AU)

(21) Appl. No.: 17/593,048

(22) PCT Filed: Mar. 6, 2020
(86) PCT No.: PCT/AU2020/050211

§ 371 Date: Sep. 7, 2021

(30) Foreign Application Priority Data
Mar. 7, 2019 (AU) 2019900757

(51) Int. Cl. A61B 5/03; A61B 5/00; A61B 5/22; A61B 5/0205

(52) U.S. Cl. CPC A61B 5/037; A61B 5/4211; A61B 5/227; A61B 2503/045; A61B 5/6847; A61B 2562/0247; A61B 2562/043; A61B 5/02055

(57) ABSTRACT

Embodiments generally relate to a device for monitoring air pressure in the body of a patient. The device comprises a tube comprising a feeding lumen; a sensor lumen positioned parallel to the feeding lumen; at least one sensor positioned in the sensor lumen; and at least one perforation positioned to expose the at least one sensor to an air pressure within the body of a patient when the device is positioned at least partially in the airway of the patient. The at least one sensor is configured to generate data related to the pressure to which the sensor has been exposed.

### CROSS-REFERENCE TO RELATED APPLICATIONS

The present application claims priority from Australian Provisional Patent Application No 2019900757 filed on 7 Mar. 2019, the contents of which are incorporated herein by reference in their entirety.

### TECHNICAL FIELD

Embodiments generally relate to systems, devices and methods for determining laryngopharyngeal and/or lower oesophageal sphincter pressure. In particular, embodiments relate to systems, devices and methods for determining the laryngopharyngeal pressure of a neonate undergoing non-invasive respiratory support by high flow nasal cannula (HFNC), continuous positive airway pressure (CPAP), or other interface in order to ultimately understand lung pressure; or for determining the lower oesophageal sphincter pressure of a neonate undergoing feeding via a gastric tube to proactively manage the risk of gastroesophageal reflux.

### BACKGROUND

Despite being necessary for life outside of the womb, a baby's lungs are some of the last structures to finish developing during pregnancy. As a result, the vast majority of infants who are born prematurely require some form of breathing support to survive.

Current non-invasive breathing support systems attempt to keep the lungs open and inflated between breaths by providing a positive air pressure or flow to the infant's respiratory system. However, due to the possibility of unaccounted leaks within the system, clinicians are forced to use a trial and error method when setting the level of respiratory support. Delivering a pressure which is too high or too low can have serious health consequences for the baby. It results in slower physical development, increased length of stay in the neonatal intensive care unit (NICU) and higher morbidity rates later in life.

It is desired to address or ameliorate one or more shortcomings or disadvantages associated with prior systems and methods for determining laryngopharyngeal and/or lower oesophageal sphincter pressure, or to at least provide a useful alternative thereto.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY OF INVENTION

Some embodiments relate to a device for monitoring air pressure in the body of a patient, the device comprising:
a tube comprising a feeding lumen;
a sensor lumen positioned parallel to the feeding lumen;
at least one sensor positioned in the sensor lumen; and
at least one perforation positioned to expose the at least one sensor to an air pressure within the body of a patient when the device is positioned at least partially within an airway of the patient;
wherein the at least one sensor is configured to generate data related to the pressure within the airway to which the sensor has been exposed.

According to some embodiments, the tube comprises the sensor lumen and the at least one sensor does not protrude from an exterior of tube.

Some embodiments further comprise a sensor conduit coupled along the length of the tube, wherein the sensor conduit comprises the sensor lumen.

In some embodiments, the air pressure is at least one of laryngopharyngeal pressure, lower oesophageal sphincter pressure, lower oesophageal pressure and lung pressure.

Some embodiments further comprise a connector portion to allow the tube to be fluidly coupled to a feeding line and to allow the at least one sensor to be electrically coupled to a processing unit.

According to some embodiments, the tube is at least one of a nasogastric or orogastric tube.

In some embodiments, the tube acts as an enteral feeding tube.

Some embodiments further comprise at least one positioning marker to assist in placing the device into at least one of the laryngopharyngeal, lower oesophageal sphincter and lower oesophageal region of the patient.

According to some embodiments, the sensor is an optic fibre pressure sensor.

In some embodiments, the sensor is Fibre Bragg Grating sensor.

According to some embodiments, the at least one sensor comprises at least two sensors positioned along a length of the tube.

In some embodiments, at least two of the at least two sensors are positioned at least 3 cm apart.

In some embodiments, the at least one sensor comprises at least two sensors positioned around a circumference of the tube.

Some embodiments further comprise a light source to shine light through an anterior section of the patient's throat to assist in positioning of the device at least partially in the airway of the patient.

In some embodiments, monitoring air pressure in the body of a patient comprises monitoring air pressure within an upper digestive tract of the patient.

Some embodiments relate to a system for monitoring airway pressure in a patient, the system comprising:
the device of some other embodiments; and
a processor unit configured to receive sensor data generated by the at least one sensor.

Some embodiments further comprise a display device.

In some embodiments, the processing device is configured to determine at least one of a pressure administered by the device, a pressure being delivered to the airway, an indication of whether the pressure being delivered is outside a predetermined limit, an indication of whether an error exists in the device, and at least one parameter relating to airflow dynamics in the airway.

According to some embodiments, the system is configured to detect airflow variations and to analyse airflow dynamics.

In some embodiments, the system is configured to detect gastroesophageal reflux.

In some embodiments, the system allows for real-time monitoring.

In some embodiments, the system is configured to determine an invalid sensor reading.

According to some embodiments, the processor unit is configured to determine at least one of a respiratory rate and a heart rate based on the sensor data.

Some embodiments relate to systems, methods and devices for determining laryngopharyngeal and lower oesophageal sphincter pressure.

Some embodiments relate to a device that acts as a gastric tube and is able to determine the laryngopharyngeal pressure and, by inference, lung pressure of a patient undergoing any form of non-invasive respiratory support. The gastric tube may be a nasogastric or orogastric tube in some embodiments. The said device incorporating multiple functions allows for clinicians to understand the actual air pressure in the lungs of a patient to accurately account for any air leakages in the respiratory support system without any additional invasiveness to the treatment.

Some embodiments of the device comprise a detection means of the laryngopharyngeal pressure through the recruitment of at least 1 sensor on the device.

Some embodiments of the device comprise a detection means of the laryngopharyngeal pressure through the recruitment of multiple sensors on the device. The incorporation of multiple sensors allows for measurement across a broader section of the respiratory tract to be able to determine a mean air pressure. The said mean value can provide a higher confidence level in the accurate representation of the true lung pressure compared to utilising only 1 sensor.

According to some embodiments, the device comprises real-time (continuous) monitoring capacity of laryngopharyngeal air pressure. This allows for instantaneous detection of changes in air pressure delivered caused by opening or closing of the mouth, positional changes of the nasal prongs, or any other means that could cause disruption, leaks and changes to the administered pressure. Continuous monitoring of air pressure delivered also allows for the respiratory support to reach the target lung pressure for the patient more quickly and efficiently.

In some embodiments, the multiple sensors on the device are arranged in an array that spans the circumference of the tube. This allows for measurement of air pressure in all possible directions and thus is also able to account for any sensors potentially occluded by surrounding anatomy, tissue and related substances.

According to some embodiments, the multiple sensors are positioned at different points along the length of the tube such that they span the laryngopharynx region of the respiratory tract, within 3 cm and preferably within a suitable distance of the center of said region. The laryngopharynx region contains the junction where the respiratory tract and esophageal tract split towards their paths to the lungs and stomach respectively, and therefore represents the closest point for the gastric tube to obtain an accurate measurement of lung pressure.

Some embodiments of the device incorporate a light source on the tube. The light source is intended to provide a visual indicator of tube placement through the correct portion of the patient's anatomy by having a light that shines through the anterior section of the throat.

According to some embodiments, the device incorporates a light source positioned at a point along the length of the tube such that it sits within the laryngopharynx region of the respiratory tract, within 3 cm and preferably within a suitable distance of the center of said region. This allows for the light source to provide a visual indicator of sensor placement at the correct portion within the laryngopharynx region of the respiratory tract to ensure that accurate measurements of lung pressure will be obtained.

In some embodiments, the multiple sensors positioned at different points along the length of the tube allow for air pressure variations along the length of the laryngopharynx and surrounding regions to be determined. This allows for analysis of airflow dynamics in the system, as airflow is governed by pressure gradients. Due to variations in patient's lung compliance, in certain cases achieving a target air pressure may not necessarily result in sufficient lung inflation. Therefore having an understanding of the resultant airflow in the system enables clinicians to appropriately adjust the administered pressure to suit the patient's needs.

According to some embodiments, the multiple sensors positioned at different points along the length of the tube can be leveraged to reveal the location of the vocal cords. The vocal cords provide an indication of appropriate sensor placement in the larynopharynx region. It is also known that the air pressure in the system will vary along the length of said region until the entry point of the stomach. Therefore establishing the location of the vocal cords allows for appropriate placement of said device.

In some embodiments, the device acts as a gastric tube and is able to measure lower oesophageal sphincter pressure and, by inference, detect gastroesophageal reflux. The said device incorporating multiple functions allows for clinicians to administer feeding while proactively being able to detect reflux without waiting for physical symptoms, such as regurgitation, to present.

In some embodiments, the device comprises of a detection means of lower oesophageal sphincter pressure through the recruitment of at least 1 sensor on the device.

According to some embodiments, the device comprises of a detection means of the lower oesophageal sphincter pressure through the recruitment of multiple sensors on the device. The incorporation of multiple sensors allows for measurement across a broader section of the upper gastrointestinal tract to be able to determine a mean oesophageal sphincter pressure. The said mean value can provide a higher confidence level in the accurate representation of the true oesophageal sphincter pressure compared to utilising only 1 sensor.

According to some embodiments, the device comprises real-time (continuous) monitoring capacity of gastroesophageal reflux. This allows for instantaneous detection of changes in the oesophageal sphincter pressure, or any other means that could cause a retrograde flow of gastric contents into the oesophagus. Continuous monitoring of gastroesophageal reflux also allows for the proactive feeding management of a patient prior to having physical symptoms, such as regurgitation, to present.

According to some embodiments, the multiple sensors on the device are arranged in an array that spans the circumference of the tube. This allows for measurement of oesophageal pressure in all possible directions and thus is also able to account for any sensors potentially occluded by surrounding anatomy, tissue and related substances.

In some embodiments, the multiple sensors are positioned further distally along the length of the tube such that they span the lower oesophageal sphincter region of the gastrointestinal tract, within a suitable distance of the center of said region. By accessing said region which is closest to the entry point of the stomach, proactive and accurate detection of gastroesophageal reflux is possible.

Some embodiments relate to a monitoring system, which comprises a connection to the gastric tube with pressure sensors, which is responsible for measuring the laryngopharyngeal pressure.

Some embodiments relate to a monitoring system, which comprises a connection to the gastric tube with pressure sensors, which is responsible for determining airflow dynamics.

Some embodiments relate to a processing unit in communication with a device according to some previously described embodiments, wherein the processing device is configured to capture data from all of the pressure sensors and account for invalid sensor readings. A sensor reading may be invalid if it is occluded by surrounding anatomy, tissue and related substances.

Some embodiments relate to a processing unit in communication with a device according to some previously described embodiments, wherein the processing device allows for determination of laryngopharyngeal pressure and, by inference, lung pressure. By virtue of measuring the air pressure in the laryngopharynx region, which is the closest point for said device to be positioned relative to the lungs, an inference of of air pressure in the lungs is possible.

Some embodiments relate to a processing unit in communication with a device according to some previously described embodiments wherein the processing unit allows for real-time (continuous) monitoring of laryngopharyngeal pressure and, by inference, lung pressure. This allows for continuous adjustment of the administered air pressure of the respiratory support to be able to reach the target lung pressure for the patient more quickly and efficiently.

Some embodiments relate to a processing unit in communication with a device according to some previously described embodiments, wherein the processing unit allows for determination of the location of the vocal cords. The vocal cords provide an indication of appropriate sensor placement in the larynopharynx region. It is also known that the air pressure in the system will vary along the length of said region until the entry point of the stomach. Therefore establishing the location of the vocal cords allows for appropriate placement of said device.

Some embodiments relate to a display unit in communication with a device according to some previously described embodiments, wherein the display unit provides a plurality of measurements to the clinicians, which may include but are not limited to: (i) the administered pressure, (ii) the pressure at the laryngopharynx, (iii) an indication of whether the pressure being delivered is outside set limits, (iv) an indication of whether an error exists in the device, and (v) the airflow dynamics in the airways.

Some embodiments relate to a monitoring system, which comprises a connection to the gastric tube with pressure sensors, which is responsible for measuring the lower oesophageal sphincter pressure.

Some embodiments relate to a processing unit in communication with a device according to some previously described embodiments, wherein the processing unit is configured to capture data from all of the pressure sensors and account for invalid sensor readings. A sensor reading may be invalid if it is occluded by surrounding anatomy, tissue and related substances.

Some embodiments relate to a processing unit in communication with a device according to some previously described embodiments, wherein the processing unit allows for determination of lower oesophageal sphincter pressure and, by inference, a detection of gastroesophageal reflux. This allows for proactive management of the gastric tube placement and feeding line to prevent gastroesophageal reflux prior to presentation of physical symptoms, such as regurgitation.

Some embodiments relate to a processing unit in communication with a device according to some previously described embodiments, wherein the processing unit allows for real-time (continuous) monitoring of lower oesophageal sphincter pressure and, by inference, gastroesophageal reflux. This allows for continuous adjustment of the gastric tube positioning or flow from feeding line to effectively prevent gastroesophageal reflux.

Some embodiments relate to a display unit in communication with a device according to some previously described embodiments, wherein the display unit provides a plurality of measurements to the clinicians, which may include but not limited to: (i) the lower oesophageal sphincter pressure, (ii) the intragastric pressure, and (iii) an indication of gastroesophageal reflux.

Some embodiments relate to a manufacturing method for the gastric pressure sensing tube, which can be made from but not limited to the following materials for the outer tubing: polyurethane, silicone, polypropylene, polyethylene, nylon.

Some embodiments relate to a manufacturing method for the gastric pressure sensing tube, which can be made from but not limited to the following materials for the internal electrical wiring: copper, stainless steel, nitinol, platinum alloys, nickel and silver plated wires.

Some embodiments relate to a manufacturing method for a device according to some previously described embodiments, which allows for co-extrusion of the tubing and wiring materials. The advantage of co-extrusion over traditional manufacturing practices such as hand stringing wires through multi-lumen tubing is that it: (i) allows for the wiring to exist within the walls of the tubing, (ii) reduces fabrication costs and scrap rates, and (iii) improves the ability to meet tight tolerances. The aforementioned materials of the outer tubing and internal electrical wiring are capable of being co-extruded.

Some embodiments relate to a manufacturing method for a device according to some previously described embodiments, which allows for creation of divot to house pressure sensor on surface of the tubing. This allows for the pressure sensor to remain flush or have minimal protrusion with respect to the outer surface of the tube to minimise the overall profile of the sensing portion of the device.

Some embodiments relate to a manufacturing method for a device according to some previously described embodiments, which allows for timed placement and connection of pressure sensor to internal electrical wiring. This allows for the pressure sensor to be placed in the correct position and attach appropriately to the internal electrical wiring.

Some embodiments relate to a manufacturing method for a device according to some previously described embodiments, which allows for timed placement and connection of proximal data transmitter to internal electrical wiring. This allows for the proximal data transmitter to be connected appropriately to the internal electrical wiring and each of the individual sensors in said device.

Some embodiments relate to a device made wherein: the tubing has a main lumen having one or more proximal connectors for connecting to a source of substances or pressure. This allows for the feeding line to remain independent of the pressure sensing aspect of said device.

Some embodiments relate to a device made wherein: the structure of the tubing and internal wiring maintains sufficient mechanical stiffness to allow for delivery of substances without impacting pressure sensing capability. This allows for the feeding line to remain active while maintaining accurate pressure measurement.

Some embodiments relate to a device made wherein: the structure of the tubing and internal wiring maintains sufficient flexibility to allow for safe navigation through anatomy.

Some embodiments relate to a method for device placement, which can be conducted per the following example: first perform a pre-placement measurement check of the relative positioning of the laryngopharynx region to the overall length of the gastric tube. Then place the distal end of the device through the patient's nose or mouth. Advance said tube, using proximal depth markings as an indication of appropriate placement relative to the patient's anatomy. Activate the light source of the gastric tube to provide a visual inspection of appropriate sensor placement relative to the patient's anatomy. The pressure measurements provided by the tube can also assist in determining the location of the vocal cords and appropriate sensor placement relative to the patient's anatomy. Then connect proximal data transmitter of tube to processing and display unit. Connect proximal inner lumen of tube to feeding line, close or vent tube.

Some embodiments relate to a method for continuous monitoring of the laryngopharynx pressure of a patient undergoing any form of non-invasive respiratory support, which can be conducted per the following example: first set administered air pressure of the non-invasive respiratory support system used. Then observe the laryngopharynx pressure measurements being provided by said device. Also observe any warnings provided by the processing and display unit, checking first for whether there is any error from the device. Compare the air pressure in the laryngopharynx versus the level of respiratory support to assess the extent of air leakage in the system. Then compare the air pressure in the laryngopharynx to a baseline level to determine whether the air pressure is outside set limits. The baseline level may be a level of pressure or flow being administered by a respiratory support machine. Observe patient respiratory activity and vital signs. Adjust administered air pressure or flow as required to achieve desired level of laryngopharyngeal air pressure for the patient.

Some embodiments relate to a method for continuous monitoring of the airflow dynamics of a patient undergoing any form of non-invasive respiratory support, which can be conducted per the following example: first set administered air pressure of the non-invasive respiratory support system used. Observe the airflow measurements being provided by said device. Then observe any warnings provided by the processing and display unit, checking first for whether there is any error from the device. Compare the airflow in the laryngopharynx versus the administered pressure to assess the extent of air leakage in the system. Then compare the airflow in the laryngopharynx to a baseline level to determine whether the airflow is outside set limits. Observe patient respiratory activity and vital signs. Adjust administered air pressure or flow as required to achieve desired airway airflow characteristics for the patient.

Some embodiments relate to a method for device placement, which can be conducted per the following example: first perform a pre-placement measurement check of the relative positioning of the lower oesophageal sphincter region to the overall length of the gastric tube. Then place the distal end of the device through the patient's nose or mouth. Advance said tube, using proximal depth markings as an indication of appropriate placement relative to the patient's anatomy. The pressure measurements provided by the tube can also assist in determining the appropriate sensor placement relative to the entry point of the stomach. Then connect proximal data transmitter of tube to processing and display unit. Connect proximal inner lumen of tube to feeding line, close or vent tube.

Some embodiments relate to a method for continuous monitoring of the incidence of gastroesophageal reflux of a patient undergoing any form of feeding via a gastric tube, which can be conducted per the following example: first place the said device and observe the lower oesophageal sphincter pressure measurements. Also observe any warnings provided by the processing and display unit, checking first for whether there is any error from the device. Compare the lower oesophageal sphincter pressure to the intragastric pressure to assess the risk of gastroesophageal reflux.

Observe patient respiratory activity and vital signs. Adjust positioning of gastric tube or flow of feeding as required to prevent gastroesophageal reflux for the patient.

### BRIEF DESCRIPTION OF DRAWINGS

Embodiments are described in further detail below, by way of example and with reference to the accompanying drawings, in which:

FIG. 1 shows side and isometric views of a gastric tube according to some embodiments, showing the location of the respiratory pressure sensors on the outer surface of the tube and how sensors are fitted onto the tube;

FIG. 2 is a sectional view of the anatomy of the respiratory tract, and a schematic diagram showing how the gastric tube of FIG. 1 fits within the anatomy and its targeting of the laryngopharynx region;

FIG. 3 is a side view of the gastric tube of FIG. 1, showing the location of depth markings to assist in appropriate placement and positioning of the sensors of the tube into the laryngopharynx region;

FIG. 4 is a perspective view of an alternative embodiment of the gastric tube that allows for a light source to be connected within the inner lumen which illuminates through the patient's throat to indicate appropriate positioning of the sensors;

FIG. 5 is a schematic view of the gastric tube of FIG. 1 or FIG. 4 that shows the internal wiring within the wall which connects the pressure sensor to the proximal data transmitter;

FIG. 6 shows side and isometric views of an alternative embodiment of the gastric tube, showing the location of the oesophageal pressure sensors and how the sensors are fitted within the diameter of the tube;

FIG. 7 is a sectional view of the gastrointestinal tract and a schematic diagram of the device of FIG. 1, 4 or 6 showing where the pressure sensors will be positioned to measure across the lower oesophageal sphincter and into the stomach;

FIG. 8 is a block diagram of a system incorporating the device of FIG. 1;

FIGS. 9A and 9B show display devices according to some embodiments;

FIG. 10 is a diagram showing the system of FIG. 8 in further detail;

FIG. 11 is a flowchart showing a method for positioning the device of FIG. 1 in the laryngopharynx region;

FIG. 12 is a flowchart showing a method for continuous monitoring of the laryngopharynx pressure of a patient;

FIG. 13 is a flowchart showing a method for continuous monitoring of the airflow dynamics of a patient;

FIG. 14 is a flowchart showing a method for positioning the device of FIG. 1 in the lower oesophageal sphincter region;

FIG. 15 is a flowchart showing a method for continuous monitoring of the incidence of gastroesophageal reflux of a patient;

FIG. 16 shows an embodiment of a sub-system comprising the device of FIG. 1;

FIG. 17 shows an alternative embodiment of a sub-system comprising the device of FIG. 1;

FIG. 18 shows a further alternative embodiment of a sub-system comprising the device of FIG. 1; and

FIG. 19 shows a further alternative embodiment of a sub-system comprising the device of FIG. 1.

### DETAILED DESCRIPTION

Embodiments generally relate to systems, methods and devices for determining laryngopharyngeal and lower oesophageal sphincter pressure are disclosed herein.

The most common lung problem in premature infants is Respiratory Distress Syndrome (RDS), which remains the most common single cause of death in the first year of a newborn's life (Copland, I et al. Understanding the mechanisms of Infant Respiratory Distress and Chronic Lung Disease. American Journal of Respiratory Cell and Molecular Biology. 2002; 26(3):261-265.).

RDS is caused by the insufficient production of pulmonary surfactant and the structural immaturity of the lungs. Surfactant serves to lower the surface tension at the alveoli surface preventing the lungs from collapsing during expiration. Due to limited surfactant production, infants with RDS have difficulty expanding their lungs, therefore preventing the exchange of oxygen and carbon dioxide from occurring (Copland, I et al. Understanding the mechanisms of Infant Respiratory Distress and Chronic Lung Disease. American Journal of Respiratory Cell and Molecular Biology. 2002; 26(3):261-265.).

Neonates suffering RDS are treated by the use of non-invasive respiratory support, however, further complications can arise as a result of the inaccuracies of this practice (Boel L, Broad K, Chakraborty M. Non-invasive respiratory support in newborn infants. Paediatrics and Child Health. 2017 Nov. 15.). Current non-invasive respiratory support mechanisms fail to accurately account for sources of air leaks in the system, which can occur at the nose, mouth and stomach. As a result, the pressure or flow set on the machine does not necessarily reflect the amount of air pressure or flow that reaches the lungs. This ambiguity means that it is possible to unknowingly over or under inflate the lungs, leading to serious health consequences for the infant. Clinicians, therefore, spend large portions of time monitoring the neonate for physical signs of distress, a burden which is amplified by the delayed presentation of symptoms.

An atelectasis is the partial or complete collapse of the lungs. For premature infants, this often occurs when the delivery of pressure to the lungs of infants with RDS is insufficient (Dargaville P Al, Tingay D G. Lung protective ventilation in extremely preterm infants. J Paediatr Child Health. 2012 September; 48(9):740-6). Symptoms of atelectasis include difficulty breathing, short and rapid breathing, increased heart rate and cyanosis (blue coloured skin). An atelectasis is generally treated with medications, physical therapy and by increasing the level of respiratory support.

A pneumothorax occurs when the pressure in the lungs is too high, causing air to burst through the lung lining and fill into the intrapleural space, potentially leading to a lung collapse (Dargaville P A, Gerber A, Johansson S, De Paoli A G, Kamlin C O, Orsini F, Davis P G. Incidence and outcome of CPAP failure in preterm infants. Pediatrics. 2016 Jul. 1; 138(1):e20153985.). Symptoms of a pneumothorax include sharp pains in the chest, difficulty breathing, short and rapid breathing and an increased heart rate. For infants born between 25-28 weeks of pregnancy, 3.7% of CPAP treatments result in a pneumothorax, which reduces to 2.7% for infants at 29-32 weeks (Dargaville P A, Gerber A, Johansson S, De Paoli A G, Kamlin C O, Orsini F, Davis P G. Incidence and outcome of CPAP failure in preterm infants. Pediatrics. 2016 Jul. 1; 138(1):e20153985.). A pneumothorax can be treated surgically but is most commonly treated by a chest drain. A chest drain can take up to three days and involves the removal of air and liquid from the intrapleural space via insertion through the chest wall (Kirmani B H, Page R D. Pneumothorax and insertion of a chest drain. Surgery (Oxford). 2014 May 1; 32(5):272-5).

Clinicians currently use a variety of methods in an attempt to accommodate for leaks in the system, most of which have focused on leaks around the mouth and nose. While leaks may also occur through the stomach, due to the high impedance of the lower oesophageal sphincter, these leaks are relatively minor in comparison and are often ignored (Mehta S, McCool F D, Hill N S. Leak compensation in positive pressure ventilators: a lung model study. European Respiratory Journal. 2001 Feb. 1; 17(2):259-67.).

Chin straps are sometimes used in an attempt to minimise the fluctuations in delivered pressure caused by the opening and closing of the infant's mouth. While some NICUs routinely use chin straps, others do not view this as an adequate solution to the need, a contention shared by a study in 2014 which found that there was no clinically significant benefit of using chin straps (Feltman D. 2014, Does routine use of chinstraps result in improved clinical outcomes for neonatal patients requiring non-invasive pressure ventilation. Paper presented at Vermont Oxford Neonatal Conference 2014. Viewed 25 Oct. 2018. <http://www.vtoxford.org/meetings/AMQC/Handouts2014/LearningFair/NorthShore_DoesRoutineUseofChinstraps.pdf>). Additionally, chin straps have several disadvantages. Forcefully keeping the mouth closed can be uncomfortable for the infant and preventing the infant from being able to freely open their mouth to yawn, burp or vomit may cause agitation.

To minimise leaks through the nostrils, namely around the nasal prongs, clinicians may try to use nasal prongs which fit firmly in the nostrils (Chen C Y, Chou A K, Chen Y L, Chou H C, Tsao P N, Hsieh W S. Quality improvement of nasal continuous positive airway pressure therapy in neonatal intensive care unit. Pediatrics & Neonatology. 2017 Jun. 1; 58(3):229-35.). However, limitations to this method exist. Firstly, the rapid growth of neonates means that well-fitting nasal prongs rarely remain well fitting, requiring a rapid turnover of the nasal prong interface, which is very expensive. The sensitivity of the neonatal nasal area also means that although prongs should ideally fit firmly to minimise leaks, the risk of nasal pressure injury must be taken into account. Nasal prongs which are too firm fitting pose the risk of skin breakdown, bruising, bleeding and in severe cases, altered nasal shape (Neonatal respiratory distress including CPAP. Queensland Clinical Guideline 2018. Viewed 25 Oct. 2018. <https://www.health.qld.gov.au/_data/assets/pdf_file/0012/141150/g-cpap.pdf>). Similar to chin straps, minimising leaks using nasal prongs only addresses one source of leakage, and comes with many limitations.

Manufacturers of non-invasive respiratory support systems have also recognised that the open circuit of the system is inherently leaky, resulting in inaccurate delivery of treatment. Recently, non-invasive respiratory support systems incorporating the ability to compensate for leaks have become available. These methods work by using external measurements of flow and resistance and adjusting the administered airflow accordingly. However, there is limited evidence on the effectiveness of leak compensatory non-invasive respiratory support systems in delivering the required level of support in the presence of air leaks. A study found that the non-invasive respiratory support systems with built-in leak compensation may be able to compensate for leaks to maintain mean CPAP levels, however do so with rather large pressure swings (Drevhammar T, Nilsson K, Zetterström H, Jonsson B. Seven Ventilators Challenged With Leaks During Neonatal Nasal CPAP: An Experimental Pilot Study. Respiratory care. 2015 Feb. 24:respcare-03718.). For example, the study found that when leaks were introduced into the system, there was a gradual compensation for the initial pressure drop, but when the leak was stopped, an overshoot of the pressure was observed. The study concluded that “leak compensation is no guarantee for a more pressure-stable system”.

To overcome the drawbacks associated with non-invasive respiratory support systems, a device that can enable the delivery of the desired pressure into the lungs of neonates is required.

Gastroesophageal Reflux (GER) is known to affect more than two-thirds of otherwise healthy infants (Lightdale J, Gremse D. Gastroesophageal Reflux: Management Guidance for the Pediatrician. Pediatrics. 2013 May; 131(5).), and is defined as the physiologic passage of gastric contents into the oesophagus. Gastroesophageal Reflux Disease (GERD) is distinguished as reflux caused by underlying symptoms or complications.

Particularly in infants, GERD is primarily caused by an insufficient development of the lower oesophageal sphincter, which in turn is unable to provide the requisite pressure to prevent retrograde flow of gastric contents into the oesophagus (Czinn S, Blanchard S. Gastroesophageal Reflux Disease in Neonates and Infants. Pediatric Drugs. 2013 February; 15(1).). Additionally diagnosis of GERD in infants can be difficult as the typical adult symptoms such as heartburn, vomiting, and regurgitation cannot be immediately assessed.

Studies have also shown that the presence of nasogastric tubes may increase the incidence of reflux in preterm infants (Peter C, et al. Influence of nasogastric tubes on gastroesophageal reflux in preterm infants: A multiple intraluminal impedance study. The Journal of Pediatrics. 2002 August; 141(2).), which creates an additional functional requirement for nasogastric tubes to proactively detect reflux. The current standard procedure for detection of GER is oesophageal pH monitoring, however this is not suitable for preterm infants because 90% of reflux incidents are non-acidic (Wenzl T G, et al. Gastroesophageal reflux and respiratory phenomena in infants: status of the intraluminal impedance technique. Journal of Pediatric Gastroenterology Nutrition. 1999; 28.). It is also known that there is a correlation between lower oesophageal sphincter pressure and the incidence of gastroesophageal reflux (Ahtaridis G et al. Lower esophageal sphincter pressure as an index of gastroesophageal acid reflux. Digestive Diseases and Science. 1981 November; 26(11).), however there are no naso or orogastric tubes that provide this type of measurement. Therefore it is necessary for naso or orogastric tubes to detect reflux by leveraging the lower oesophageal sphincter pressure.

Some described embodiments relate to a device which is configured to provide real time monitoring of the laryngopharynx pressure of an infant, without adding any invasiveness. The device may be designed to integrate seamlessly with existing support systems to improve the accuracy of air pressure delivery to infants on non-invasive respiratory support and allow clinicians to be proactive in their treatment.

Specifically, some embodiments relate to a device that acts as a gastric feeding tube used to administer substances directly into a patient's stomach. The device is able to determine the laryngopharyngeal pressure and, by inference, lung pressure of a patient undergoing any form of non-invasive respiratory support. The said device incorporating multiple functions allows for clinicians to understand the actual air pressure in the lungs of a patient to accurately account for any air leakages in the respiratory support system without any additional invasiveness to the treatment.

Some described embodiments relate to a device which is configured to provide real time monitoring of the pressure of the upper digestive tract of a patient.

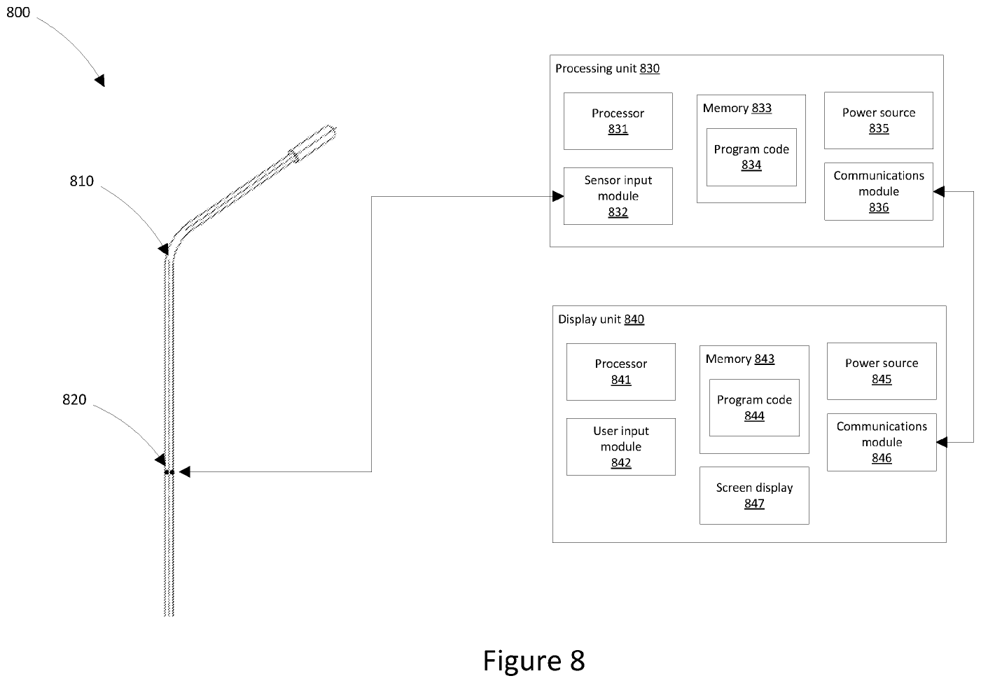

FIG. 8 shows a system 800 for determining laryngopharyngeal and lower oesophageal sphincter pressure according to some embodiments. According to some embodiments, system 800 may also be used for determining lower oesophageal pressure. According to some embodiments, system 800 may also be used for determining vital signs, such as respiration rate and heart rate. According to some embodiments, system 800 may also be used to determine core body temperature.

System 800 includes a gastric tube 810, which may be configured to act as an enteral feeding device. Tube 810 incorporates one or more sensors 820, which may comprise an array of sensors 820. According to some embodiments, sensors 820 may comprise pressure sensors, and may be fibre optic pressure sensors.

Where system 800 is being used to determine a core body temperature of a patient, sensors 820 may comprise temperature sensors, which may be fibre optic temperature sensors in some embodiments. For example, sensors 820 may be Fiber Bragg Grating (FBG) based sensors, being intrinsic sensors operating based on the wavelength modulation principle. Specifically, the sensors may work on the principle that certain wavelengths that satisfy the Bragg condition are reflected at certain positions, while all other wavelengths are reflected. This is achieved by creating gratings inside the core of an optical fibre. When the temperature of the optical fibre changes both the spacing between the gratings and the refractive index will change. Therefore, any change in temperature will cause a shift in the reflected wavelength. According to some embodiments, sensors 820 may comprise crystals such as gallium arsenide crystals mounted on the end of an optical fibre. A broadband light source may be coupled into the fibre and impinges on the crystal. The crystal behaves like a temperature sensitive cut off filter in which the crustal absorbs some light and transmits other light. The characteristic edge or transition wavelength between the reflected and transmitted spectrum is directly related to the band gap energy and hence the absolute temperature.

Sensors 820 may be positioned on an outer surface of tube 810 in some embodiments. In some alternative embodiments, sensors 820 may be positioned within the tube. According to some embodiments, sensors may be positioned along the length of tube 810, as well as around the circumference of tube 810. Sensors 820 may be optic fibre pressure sensors in some embodiments. Where pressure sensors 820 are optic fibre pressure sensors, a single optic fibre may comprise multiple sensor points along its length.

Sensors 820 can be positioned within the laryngopharynx region of the respiratory tract when tube 810 is located in the laryngopharynx region of the respiratory tract of a patient, allowing for system 800 to measure deep oropharyngeal airway pressure and subsequently understand the lung pressure of the patient. In some embodiments, tube 810 may be configured so that pressure sensors 820 are positioned in the lower oesophageal region in use, so that system 800 can be used for determining lower oesophageal pressure. In particular, this arrangement may be used to detect gastroesophageal reflux. Gastroesophageal reflux occurs when there is an abrupt decrease in lower oesophageal pressure compared to intragastric pressure. According to some embodiments, system 800 may be configured to determining lower oesophageal pressure, and generate an alarm to alert clinicians when an abrupt decrease in lower oesophageal pressure compared to intragastric pressure is detected.

The sensors 820 of the gastric tube 810 are connected to a data processing unit 830. Data processing unit 830 comprises a processor 831 and a memory 833 storing program code 834 that is executable by processor 831. Data processing unit 830 further comprises a sensor input module 832 to receive data from sensors 820, a power source 835, and a communications module 836. Communications module 836 may be configured to facilitate wired or wireless communication between data processing module 830 and other electronic devices.

In the illustrated embodiment, data processing unit 830 communicates with a display unit 840 that provides readings of: (i) the administered pressure, (ii) the pressure being delivered to the airways and lungs, (iii) an indication of whether the pressure being delivered is outside set limits, (iv) an indication of whether an error exists in the device, and (v) the airflow dynamics in the airways. According to some embodiments, display unit 840 may also be configured to display alarms and historical data of pressure measurements over time.

According to some embodiments, display unit 840 also comprises a processor 841 and a memory 843 storing program code 844 that is executable by processor 841. Display unit 840 may further comprises a user input module 842 to receive user input data, a power source 845, and a communications module 846. Communications module 846 may be configured to facilitate wired or wireless communication between display module 840 and other electronic devices, such as processing unit 830. Display unit 840 further comprises a screen display 847 that allows for data to be displayed to a user.

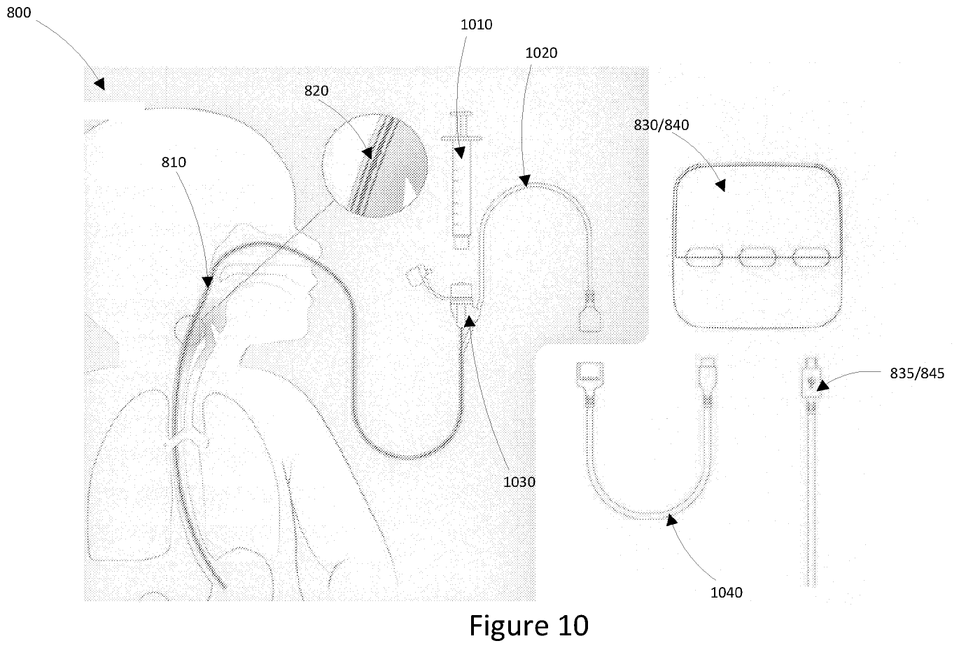

According to some embodiments, processing unit 830 and display unit 840 may be part of a single device, as shown below with reference to FIG. 10.

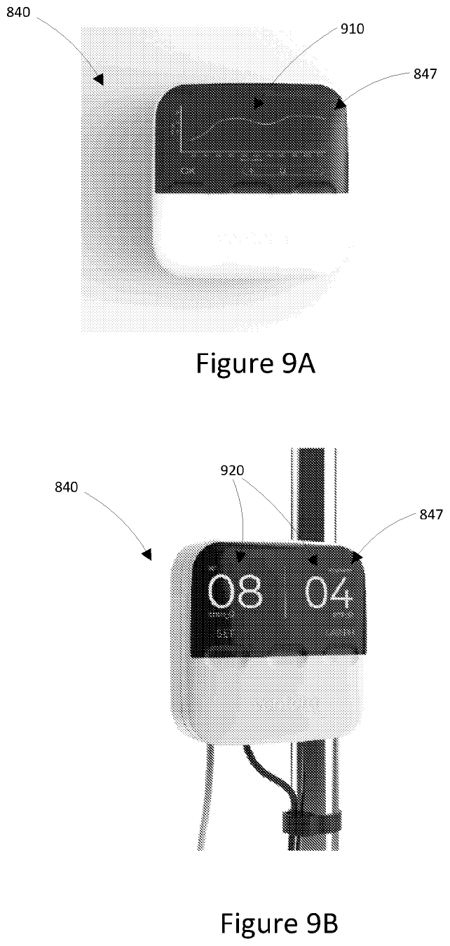

FIGS. 9A and 9B show example embodiments of display unit 840. FIG. 9A shows an example display unit 840 having a screen display 847 that is depicting measured pressure data as a graph 910. FIG. 9B shows an example display unit 840 having a screen display 847 that is depicting measured pressure data as numerical values 920.

According to some embodiments, tube 810 acts as a gastric tube and is able to measure lower oesophageal sphincter pressure and, by inference, detect gastroesophageal reflux. Tube 810 may incorporate multiple functions that allow for clinicians to administer feeding while proactively being able to detect reflux without waiting for physical symptoms, such as regurgitation, to present.

According to some alternative embodiments, gastric tube 810 incorporates an array of sensors 820 on its outer surface that can be positioned within the lower oesophageal sphincter region of the gastrointestinal tract when tube 810 is located in the lower oesophageal sphincter region of the gastrointestinal tract of a patient, allowing for system 800 to measure and subsequently detect gastroesophageal reflux. The sensors 820 of the gastric tube 810 are connected to a data processing unit 830. The data processing unit 830 communicates with a display unit 840 that provides readings of: (i) the lower oesophageal sphincter pressure, (ii) the intragastric pressure, and (iii) an indication of gastroesophageal reflux.

According to some embodiments, system 800 may also be used for determining vital signs, such as respiration rate and heart rate. Respiratory rate may be determined by processor 830 counting peaks/troughs in the airway pressure signal generated by sensors 820. Heart beats may be detected by processor 830 as an artefact of the pressure signal generated by sensors 820, and may have a unique waveform. Processor 830 may be configured to extract these waveforms from the pressure signal and process that information to measure and report heart rate.

FIG. 10 shows system 800 in further detail. FIG. 10 shows device 810 in position in the laryngopharynx region of the respiratory tract of a patient, with sensors 820 positioned to measure laryngopharyngeal pressure. A distal end of device 810 is connected to a junction 1030, which allows for device 810 to be connected to a syringe 1010. Junction 1030 also allows for sensors 820 of device 810 to be connected to combined processing and display unit 830/840 via connection cables 1020 and 1040. Cable 1020 may be designed as a consumable cable, while cable 1040 may be designed to be a reusable cable. Device 830/840 may be powered by a power supply 835/845 in the form of a power cable plugged into mains power. According to some embodiments, parts 810, 820, 1010, 1020 and 1030 of system 800 may be consumable or disposable one time use items, while parts 830/840, 835/845 and 1040 may be reusable. According to some embodiments, sensors 820 may be reusable, and may be removable from device 810, washable, and able to be re-inserted into a new device 810.

Further embodiments will now be described to provide an overall understanding of the principles of the structure, function, and use of the systems, methods and devices disclosed herein. Examples of these embodiments are illustrated in the accompanying drawings.

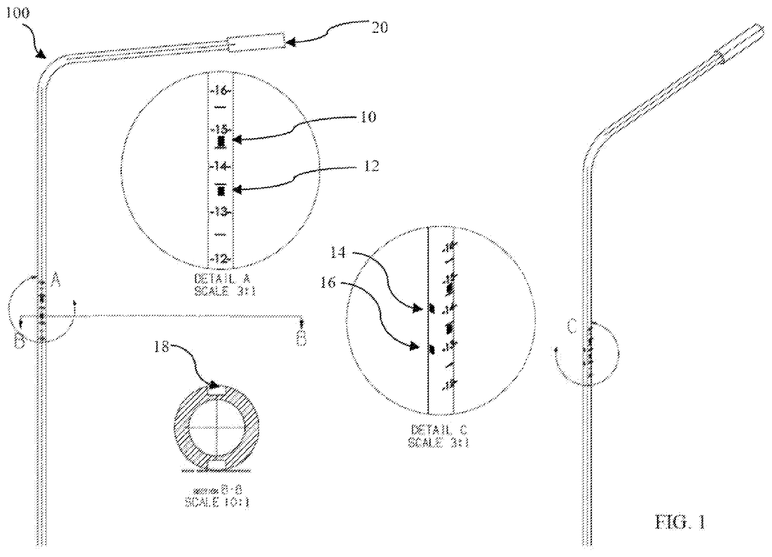

FIG. 1 shows side and isometric views of the gastric tube, showing the location of the respiratory pressure sensors on its outer surface and how the sensors are fitted onto the tube.

FIG. 1 illustrates a gastric tube 100 according to some embodiments, which is shown to include an array of pressure sensors 10, 12, 14, 16 on its outer surface arranged in a 360° spectrum to facilitate measurement in all directions. According to some embodiments, tube 100 may include the same features as tube 810, and sensors 10, 12, 14 and 16 may include the same features as sensors 820. The pressure sensors 10, 12, 14 and 16 are fitted and adhered within groove 18 so that the sensors 10, 12, 14 and 16 remain flush with the outer surface of the tube 100 or protrude minimally with respect to the outer surface of tube 100. Additionally the gastric tube 100 incorporates a larger diameter proximal connector portion 20, which serves to facilitate connection to feeding lines, consistent with standard gastric tubes, but also incorporates electrical connection to allow for power to be supplied to the pressure sensors 10, 12, 14, 16. Proximal connector 20 is described below in further detail with reference to FIGS. 10 and 12 to 15. Tube 100 may be configured so that proximal connector 20 always remains external to the patient during use. While the illustrated embodiment shows a configuration of four pressure sensors 10, 12, 14, 16, it will be appreciated that the gastric tube 100 can include any arrangement of at least one pressure sensor 10, 12, 14, 16 on its outer surface to obtain the necessary data. The purpose of including multiple pressure sensors 10, 12, 14, 16 that measure directly at the target site allows for the overall system 800 to account for any potential sensor occlusion due to the surrounding anatomy or substances so that an accurate measurement of the pressure is achieved.

A method and system for detecting the respiratory profile of a patient undergoing non-invasive respiratory support is described in International Application Number PCT/IB2017/055258, filed on Sep. 1, 2017, which is hereby incorporated by reference herein. The main purpose of the aforementioned is to provide a method and system for the detection of respiratory flow, the parameters associated therewith, and the resultant respiratory mechanics in patients undergoing the aforementioned treatment. An aspect of the aforementioned requires the recruitment of a pharyngeal catheter to measure pharyngeal pressure and an esophagus-gastric catheter to measure esophageal pressure. The pharyngeal catheter experiences a change in electrical resistance in the presence of air-flow and requires to be paired with an external pressure transducer to deduce pharyngeal pressure. The oesophagus-gastric catheter incorporates an expandable balloon at its distal tip and also requires to be paired with an external pressure transducer to deduce oesophageal pressure. The main disadvantages associated with expandable balloon tipped catheters is that they do not allow for continuous monitoring of the pressure and require periodic patency check to ensure appropriate functioning.

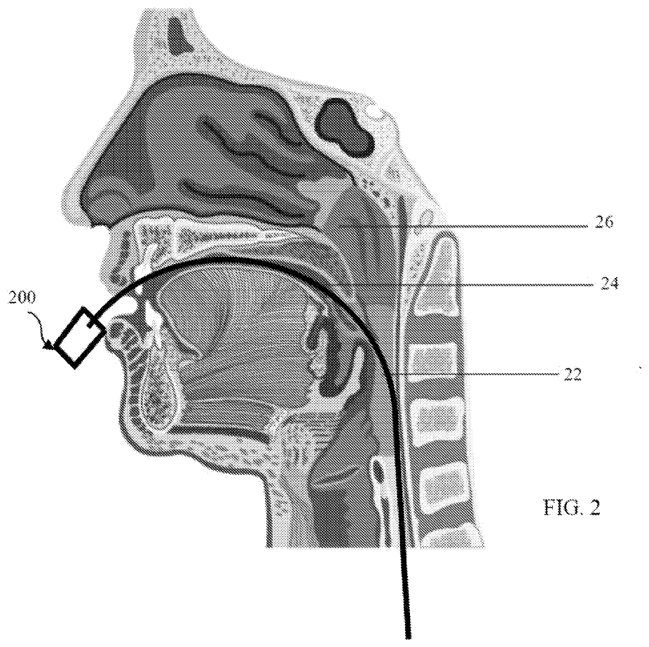

The device 100 shown in FIG. 1 may provide an improvement over the aforementioned combined use of a pharyngeal catheter and esophageal catheter as it incorporates pressure sensors 10, 12, 14, 16 measuring directly at the target site as opposed to indirect deduction of the pressure through the use of external transducers. FIG. 2. shows device 100 positioned in a target anatomical site, which may be laryngopharynx region 22 in some embodiments. The target anatomical site of the laryngopharynx region 22 as shown in FIG. 2 represents a better location for obtaining an indication of the actual pressure being delivered to the lungs compared to targeting the overall pharyngeal region, as it is positioned closer to the opening of the trachea than merely targeting the overall pharyngeal region, which potentially encompasses the nasopharynx 26 and oropharynx 24.

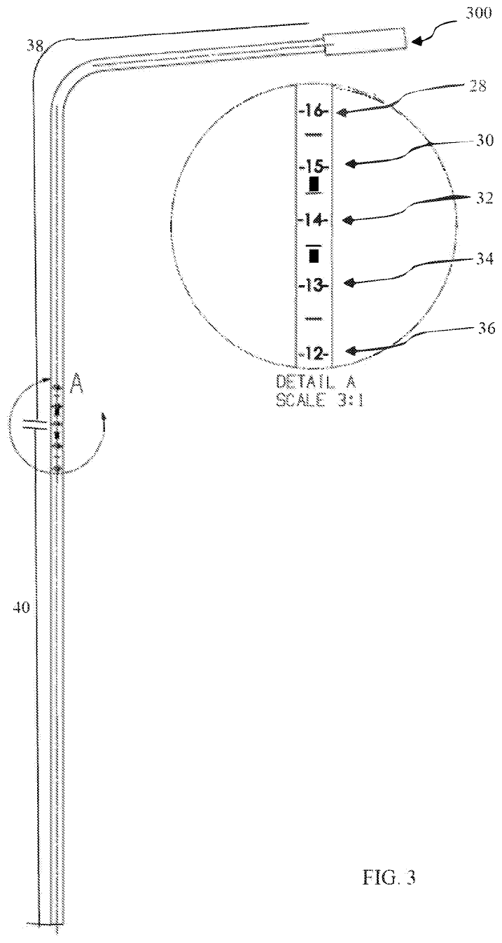

FIG. 3 shows a side view of tube 100, illustrating the location of markings that assist in appropriate placement and positioning of the sensors of the tube 100 into the laryngopharynx region 22. In order to ensure appropriate positioning of the pressure sensors in the laryngopharynx region 22, the gastric tube 100 incorporates depth markings 28, 30, 32, 34, and 36 as shown in FIG. 3, which is consistent with current clinical practice for placing gastric tubes. The depth markings 28, 30, 32, 34 and 36 also serve to delineate the position of sensors 10, 12, 14, 16 relative to the laryngopharynx region 22.

Previous studies have shown that it is possible to estimate the depth from the mouth to the mid trachea in neonates using the following weight-based formula:

Depth to Mid Trachea (cm)=Weight (kg)+6 (Eqn. 1)

(MacDonald M G, Ramasethu J, Rais-Bahrami K. 2012. Atlas of Procedures in Neonatology, 5th Edition. Lippincott, Wiliams and Wilkins, Philadelphia.).

Another study determined that the average length of a neonatal trachea is 4 cm (Wheeler D, Wong H, Shanley T. Pediatric Critical Care Medicine: Basic Science and Clinical Evidence. London: Springer; 2007.). Combining the information from the aforementioned studies establishes the following weight-based formula to locate the laryngopharynx from the mouth by subtracting half the trachea length from the original equation, which yields:

Depth to Laryngopharynx (cm)=Weight (kg)+4. (Eqn. 2)

Relative to the positioning of the stomach, it is also known that placement of gastric tubes typically relies on another weight-based formula:

Depth to Stomach (cm)=3×Weight (kg)+12 (Eqn. 3)

(Freeman D, Saxton V, Holberton J. A Weight-Based Formula for the Estimation of Gastric Tube Insertion Length in Newborns. Advances in Neonatal Care. 2012; 12(3):179-182.). Combining Eqn. 3 with Eqn. 2 reveals that the depth of the laryngopharynx is ⅓ of the depth to the stomach. Therefore, embedding the sensors 10, 12, 14, 16 at this particular section along the gastric tube 100, shown by length dimensions 38 and 40, ensures correct positioning of the sensors 10, 12, 14, 16 making up the laryngopharyngeal pressure measurement portion of the device 100.

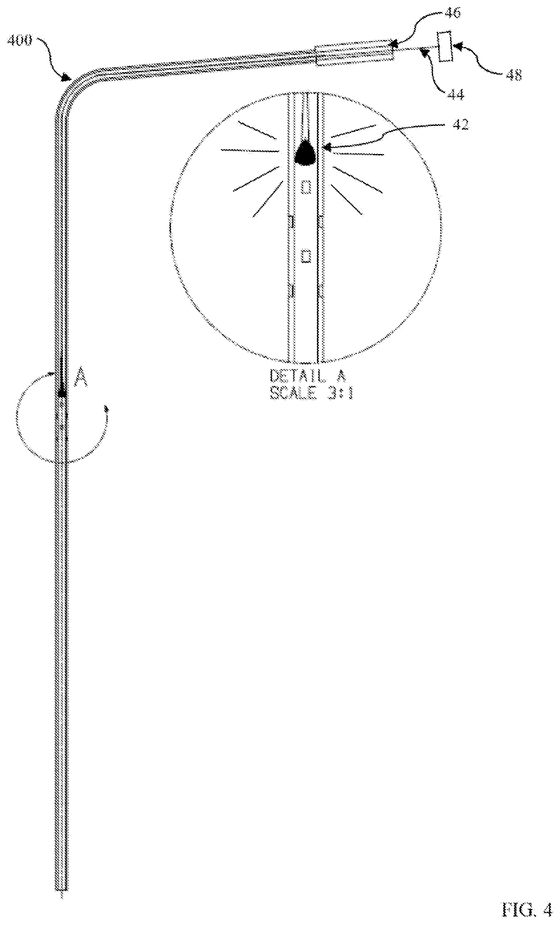

FIG. 4 shows an alternative embodiment of device 810. Device 400 as shown in FIG. 4 allows for the inner lumen 46 of the gastric tube to be connected to a light source 42 that can illuminate through the patient's throat while the tube 400 is in the respiratory tract of the patient to provide visual confirmation of appropriate sensor positioning. The light source 42 is connected to an independent wire 44 that provides electrical power and the necessary mechanical stiffness to guide the light source 42 through the entry point of the inner lumen 46 until the light 42 advances to the appropriate position along the tube 400. A depth stop 48 connected to the wire 44 controls the final resultant position of the light source 42 relative to the pressure sensors 10, 12, 14, 16. Once the light source 42 has been assembled to the gastric tube 100 it is ready for subsequent insertion into the patient.

The proposed gastric tube 810/100/400 can have an outer diameter as small as 1.5 mm for appropriate application in neonatal respiratory support. Therefore it is necessary for the embedded pressure sensors 10, 12, 13, 14 to be able to securely fit within relatively thin wall sections and incorporate minimal profile thickness. Pressure sensors as disclosed in U.S. Patent Application Publication No. 2005/0160823, filed on Dec. 28, 2004, describe designs of microfabricated piezoelectric pressure sensors available in sizes as small as 0.5 mm×0.5 mm×0.1 mm, which would fit within the wall section of the gastric tube, as shown in groove 18 per FIG. 1. One of the main features of the aforementioned pressure sensors is the high resistance to drift, which allows for stable and accurate measurements for long term implantation applications. Since gastric tubes are replaced on average every week it is necessary for the pressure sensors to be able to sustain accurate measurement during this period.

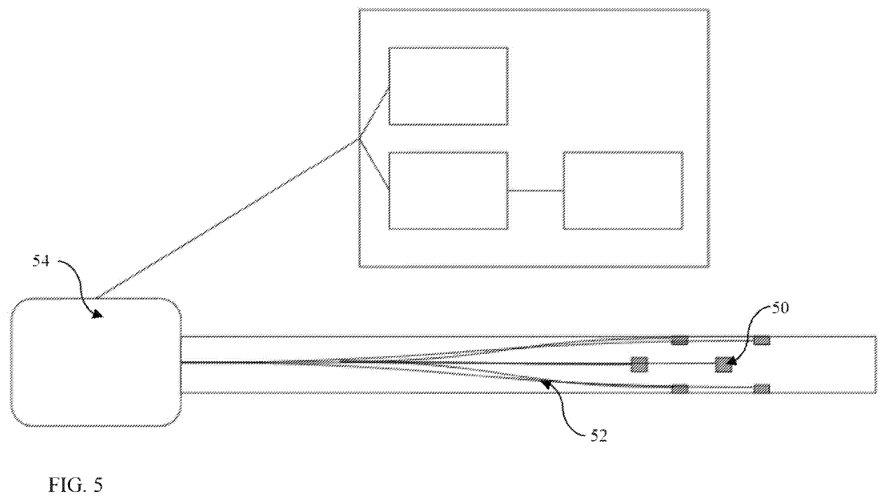

FIG. 5 shows the internal wiring within the wall of device 810/100/400. The inner wall of the gastric tube 810/100/400 incorporates electrical wiring 52 that connects each of the individual pressure sensors 50 to a proximal data transmitter 54 located near the main connection port of the tube 810/100/400, as shown in FIG. 5. Pressure sensors 50 may comprise sensors 10, 12, 14, 16. This allows for the wiring 52 to remain insulated from the patient and from the internal lumen responsible for delivering feeding substances to the stomach. The proximal data transmitter 54 is then connected to a data processing unit 830, via direct wiring or wireless means.

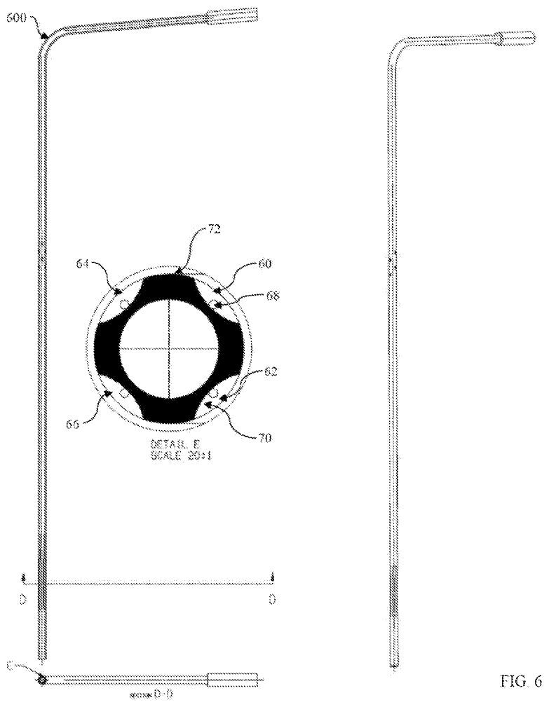

FIG. 6 illustrates an alternative tube 600, which is shown to include an array of pressure sensors 60, 62, 64, 66 integrated within the diameter of the tube 600. Pressure sensors 60, 62, 64, 66 may be located anywhere along the distal end of tube 600, and may be positioned to allow sensors 60, 62, 64, 66 to measure the lower oesophageal sphincter pressure of a patient, for example. According to some embodiments, sensors 60, 62, 64, 66 may be positioned 5 cm proximal of the distal top, for example. Sensors 60, 62, 64, 66 may be positioned in a 360° spectrum to facilitate measurement in all directions. Unlike the pressure sensors 10, 12, 14, 16 used to determine laryngopharyngeal pressure that respond to changes in respiratory flow, the design and configuration of the pressure sensors 60, 62, 64, 66 to measure the lower oesophageal sphincter pressure must be able to respond to mechanical changes in muscular tension. Traditional oesophagus-gastric catheters typically incorporate an expandable balloon near its distal tip in order to measure this mechanical pressure, however the main disadvantages associated with expandable balloon tipped catheters is that they do not allow for continuous monitoring, require periodic patency check, and can become fairly bulky and difficult to place in the patient.

Each pressure sensor 60, 62, 64, 66 on the device 600 shown in FIG. 6 incorporates a series of Fibre Bragg Grating (FBG) sensors suspended along an optical fibre 68, which is encased in a silicone member 70, that effectively acts as a strain gauge. The inner wall 72 of the gastric tube 600 serves as a rigid backing for the optical fibre 68 while the silicone encasing 70 serves as a flexible member to apply forces against the FBG sensors 60, 62, 64, 66 on the optical fibre 68. Studies have shown that it is possible to leverage optical fibre based technology to measure the muscular pressure associated with peristalsis (Arkwright J W et al. Design and clinical results from a fibre optic manometry catheter for oesophageal motility studies. Proceedings of SPIE—The International Society for Optical Engineering. 2008; 7004(70042D-1).) However in order to measure the lower oesophageal sphincter pressure and to subsequently provide an indication of gastroesophageal reflux, the configuration of the sensors 60, 62, 64, 66 relative to the target anatomical site becomes critical.

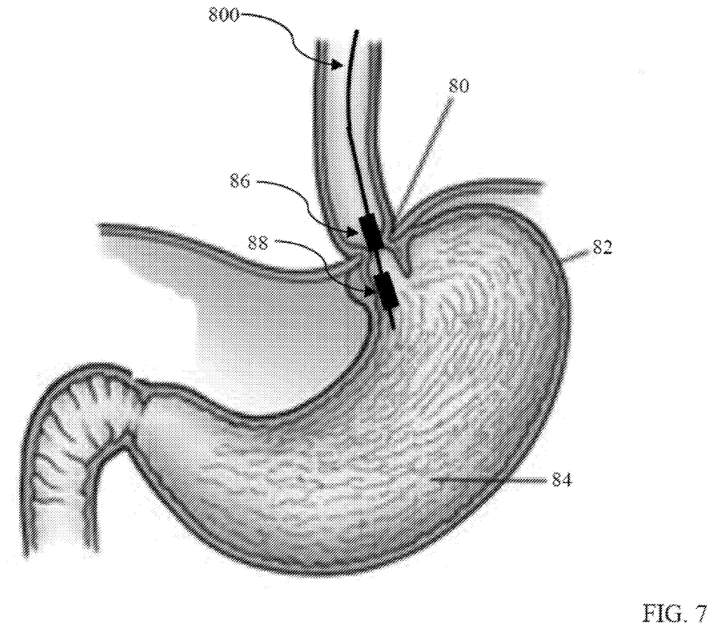

While FIG. 6 shows a configuration of four pressure sensors 60, 62, 64, 66, it will be appreciated that the gastric tube 600 can include any arrangement of at least one pressure sensor 60, 62, 64, 66 to obtain the necessary data. The purpose of including multiple pressure sensors 60, 62, 64, 66 allows for the overall system 800 to measure pressure changes along the length of the lower oesophageal sphincter 80 and across the junction into the stomach 82 as shown in FIG. 7. It is known that gastroesophageal reflux disease can be characterized by a sudden decrease in lower oesophageal sphincter pressure compared to the intragastric pressure of the stomach (Czinn S, Blanchard S. Gastroesophageal Reflux Disease in Neonates and Infants. Pediatric Drugs. 2013 February; 15(1).), which will cause any fluids in the stomach 84 to reverse flow back up the oesophagus. Therefore by building this relative measurement capability by having sensors 60, 62, 64, 66 located across positions 86 and 88 onto a gastric tube 600 it is possible to proactively detect the occurrence of gastroesophageal reflux prior to physical symptoms manifesting.

The primary objective of the data processing unit 830 as described above with reference to FIG. 8 is to capture the data from all of the pressure sensors 10, 12, 14, 16 or 60, 62, 64, 66 and produce a single pressure reading to be displayed to the user via display unit 840. In essence, when used with an embodiment of the device 810/100/400/600 that incorporates multiple pressure sensors 10, 12, 14, 16 or 60, 62, 64, 66, the system 800 is designed to require acceptable readings from at least two sensors 10, 12, 14, 16 or 60, 62, 64, 66 in order to be able to compare relative differences as a filter for data acceptability. If the difference between the readings exceeds the error range of an individual sensor, then the system 800 will be unable to deduce the correct reading and display a device error. For instance if the sensors 10, 12, 14, 16 or 60, 62, 64, 66 used have a variability of ±0.5 cmH2O, an individual error range of 1 cmH2O exists for each sensor. Conversely if the difference between the readings falls within the error range of an individual sensor, then the system 800 computes an average of said readings in order to output a single pressure value to be displayed to the user via display unit 840.

Where laryngopharyngeal pressure is being monitored, display unit 840 will be configured to provide continuous updates from the respiratory sensing element or tube 810/100/400/600 of the system 800 to the user in both graphical and numerical representation. The display unit 840 will provide critical measurements to the clinicians, not limited to but namely: (i) the administered pressure, (ii) the pressure at the laryngopharynx, (iii) an indication of whether the pressure being delivered is outside set limits, (iv) an indication of whether an error exists in the device, and (v) the airflow dynamics in the airways. With this information, the clinician will be able to make an informed decision on how the administered pressure should be varied to achieve the appropriate lung pressure for the patient.

Where lower oesophageal sphincter pressure is being monitored, display unit 840 will be configured to provide continuous updates from the oesophageal sensing element or tube 810/600 of the system 800 to the user in both graphical and numerical representation. The display unit will provide critical measurements to the clinicians, not limited to but namely: (i) the lower oesophageal sphincter pressure, (ii) the intragastric pressure, and (iii) an indication of gastroesophageal reflux.

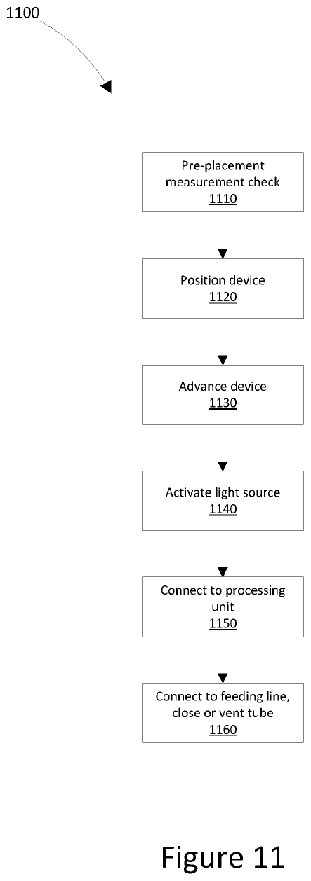

FIG. 11 shows a flowchart illustrating a method 1100 for device placement, being a positioning of the laryngopharynx region 810/100/400/600. Method 1100 can be conducted per the following steps. At step 1110, a pre-placement measurement check of the relative positioning of the laryngopharynx region to the overall length of the gastric tube 810/100/400/600 is performed. This may be performed manually by a clinician measuring from the tip of the nose of the patient to the bottom of the earlobe of the patient, and from the bottom of the earlobe of the patient to the observed midpoint between the xiphoid process and the umbilicus of the patient. At step 1120, the distal end of the device 810/100/400/600 is placed through the patient's nose or mouth. At step 1130, tube 810/100/400/600 is advanced, using proximal depth markings 28, 30, 32, 34, 36 as an indication of appropriate placement of tube 810/100/400/600 relative to the patient's anatomy. At step 1140, light source 42 of the gastric tube 810/100/400/600 is activated to provide a visual inspection of appropriate sensor placement relative to the patient's anatomy. The pressure measurements provided by the tube can also assist in determining the location of the vocal cords and appropriate sensor placement relative to the patient's anatomy. At step 1150, proximal data transmitter 54 of tube 810/100/400/600 is connected to processing unit 830 and display unit 840. At step 1160, the proximal inner lumen of tube 810/100/400/600 is connected to the feeding line, close or vent tube.

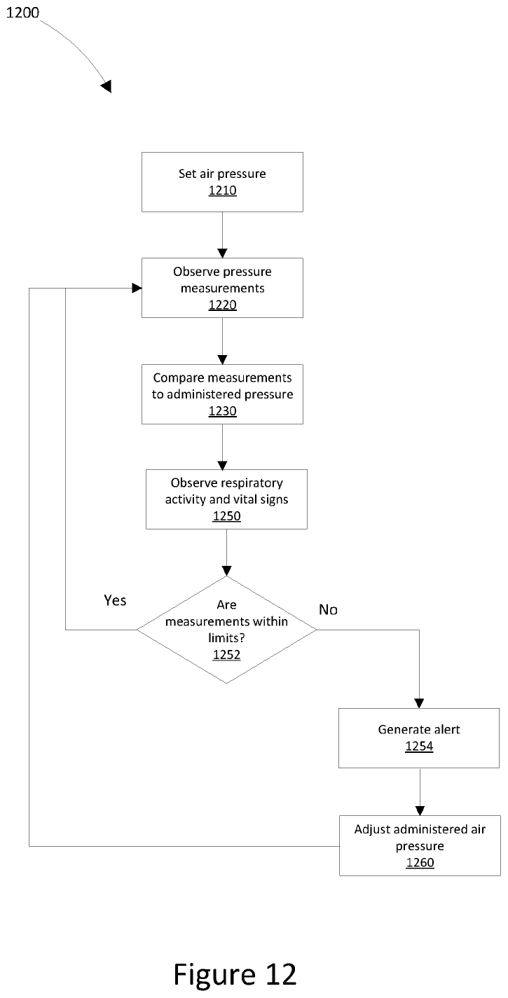

FIG. 12 shows a flowchart illustrating a method 1200 for continuous monitoring of the laryngopharynx pressure of a patient undergoing any form of non-invasive respiratory support, which can be conducted per the following steps. At step 1210, an administered air pressure of the non-invasive respiratory support system used is set.

The initial pressure may be set according to clinical guidelines, and may be set to 5-8 cmH2O in some embodiments. At step 1220, the laryngopharynx pressure measurements being provided by device 810/100/400/600 are observed. At the same time, any warnings provided by the processing unit 830 and display unit 840 are observed, checking first for whether there is any error from the device 810/100/400/600. At step 1230, the air pressure in the laryngopharynx as measured by device 810/100/400/600 versus the level of respiratory support in terms of the pressure or flow level set on a respiratory support machine is compared to assess the extent of air leakage in the system 800.

As the pressure in the laryngopharynx as measured by device 810/100/400/600 will vary based on the anatomy of the patient and the patient's condition, the set limits may be adjustable from patient to patient. At step 1250, patient respiratory activity and vital signs are observed. This may be performed manually by a clinician, or may be performed by an automatic feedback system.

At step 1252, processing unit 830 determines whether the measurements are within predetermined limits, based on the pressure or flow level set on a respiratory support machine. If the pressure or flow rate are outside the predetermined limits, this may indicate that there are leaks in the system or that the support machine is malfunctioning. In this case, at step 1254 an alert is generated to be delivered to the clinicians.

According to some embodiments, this may be delivered via display device 840. At step 1260, administered air pressure or flow is adjusted as required to achieve desired level of laryngopharyngeal air pressure for the patient. Processor 830 then continues executing the method from step 1220, by continuing to observe pressure measurements as generated by sensors 810.

If at step 1252 processing unit 830 determines that the measurements are within predetermined limits, processor 830 continues executing the method from step 1220, by continuing to observe pressure measurements as generated by sensors 810.

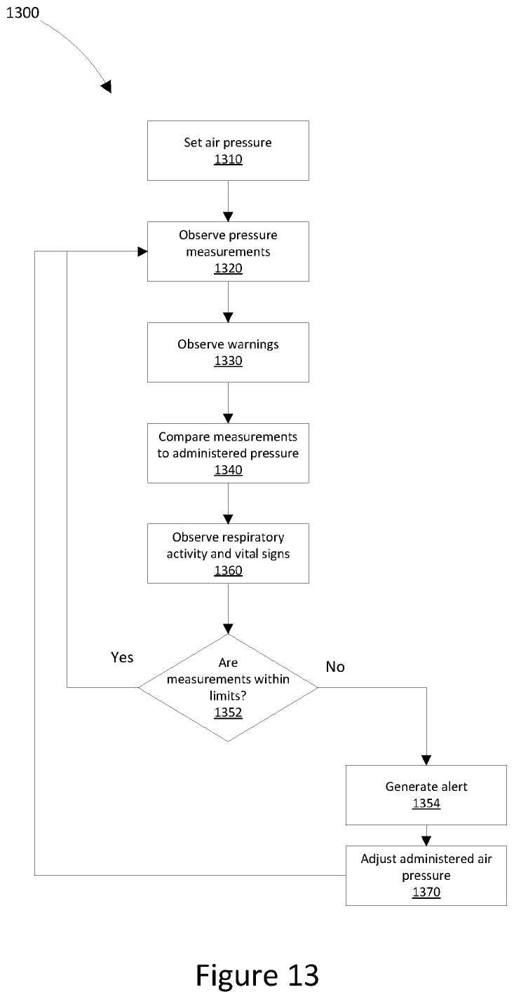

FIG. 13 shows a flowchart illustrating a method 1300 for continuous monitoring of the airflow dynamics of a patient undergoing any form of non-invasive respiratory support, which can be conducted per the following steps. At step 1310, an administered air pressure of the non-invasive respiratory support system used is set. At step 1320, the airflow measurements being provided by device 810/100/400/600 are observed. At step 1330, any warnings provided by the processing device 830 and display unit 840 are observed, checking first for whether there is any error from the device 810/100/400/600. At step 1340, the airflow in the laryngopharynx as measured by device 810/100/400/600 versus the administered pressure is compared to assess the extent of air leakage in the system 800. At step 1350, the airflow in the laryngopharynx as measured by device 810/100/400/600 is compared to a baseline level to determine whether the airflow is outside set limits. At step 1360, patient respiratory activity and vital signs are observed.

At step 1352, processing unit 830 determines whether the measurements are within predetermined limits, based on the pressure or flow level set on a respiratory support machine. If the pressure or flow rate are outside the predetermined limits, this may indicate that there are leaks in the system or that the support machine is malfunctioning. In this case, at step 1354 an alert is generated to be delivered to the clinicians. According to some embodiments, this may be delivered via display device 840.

At step 1370, the administered air pressure or flow is adjusted as required to achieve desired lung airflow characteristics for the patient. Processor 830 then continues executing the method from step 1320, by continuing to observe pressure measurements as generated by sensors 810.

If at step 1352 processing unit 830 determines that the measurements are within predetermined limits, processor 830 continues executing the method from step 1320, by continuing to observe pressure measurements as generated by sensors 810.

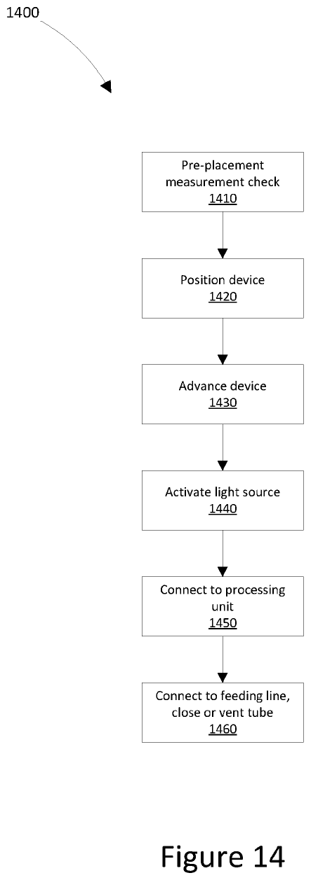

FIG. 14 shows a flowchart illustrating a method 1400 for device placement, being a method for correctly positioning device 810/100/400/600. Method 1400 can be conducted per the following steps. At step 1410, a pre-placement measurement check of the relative positioning of the lower oesophageal sphincter region to the overall length of the gastric tube 810/100/400/600 is performed. At step 1420, the distal end of the device 810/100/400/600 is placed through the patient's nose or mouth. At step 1430, tube 810/100/400/600 is advanced, using proximal depth markings 28, 30, 32, 34, 36 as an indication of appropriate placement relative to the patient's anatomy. The pressure measurements provided by the tube 810/100/400/600 can also assist in determining the appropriate sensor placement relative to the entry point of the stomach. At step 1440, the proximal data transmitter of tube 810/100/400/600 is connected to processing unit 830 and display unit 840. At step 1450, the proximal inner lumen of tube 810/100/400/600 is connected to the feeding line, close or vent tube.

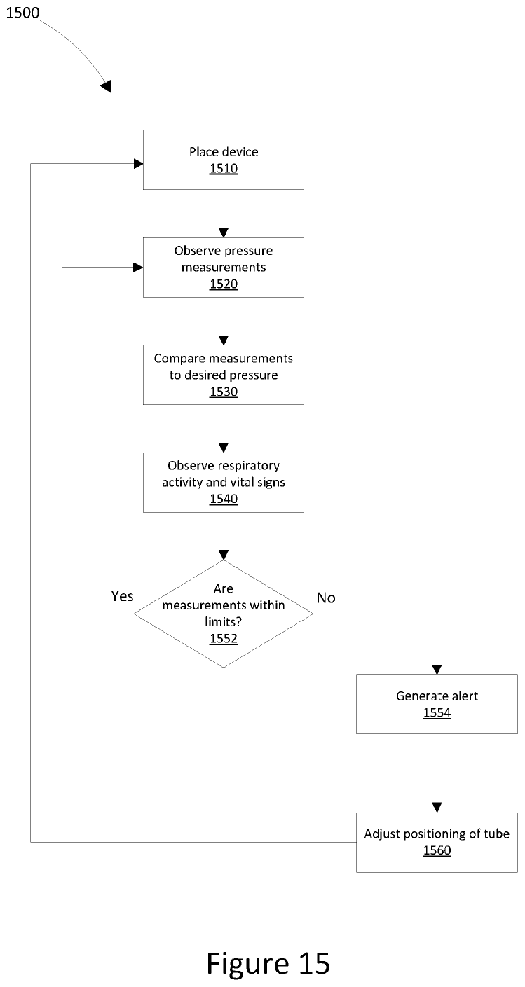

FIG. 15 shows a flowchart illustrating a method 1500 for continuous monitoring of respiratory activity of a patient undergoing any form of feeding via a gastric tube 810/100/400/600, which can be conducted per the following steps. At step 1510, device 810/100/400/600 is placed in the lower oesophageal sphincter as described above with reference to FIG. 14. At step 1520, the lower oesophageal sphincter pressure measurements are observed. At the same time, any warnings provided by the processing unit 830 and display unit 840 are observed, checking first for whether there is any error from the device 810/100/400/600. At step 1530, the lower oesophageal sphincter pressure as measured by device 810/100/400/600 is compared to the desired intragastric pressure to assess the risk of gastroesophageal reflux. At step 1540, patient respiratory activity and vital signs are observed.

At step 1552, processing unit 830 determines whether the measurements are within predetermined limits,. If the pressure is outside the predetermined limits, at step 1354 an alert is generated to be delivered to the clinicians. According to some embodiments, this may be delivered via display device 840.

At step 1540, the positioning of gastric tube 810/100/400/600 or the flow of feeding is adjusted as required to prevent gastroesophageal reflux for the patient. Processor 830 then continues executing the method from step 1520, by continuing to observe pressure measurements as generated by sensors 810.

If at step 1552 processing unit 830 determines that the measurements are within predetermined limits, processor 830 continues executing the method from step 1520, by continuing to observe pressure measurements as generated by sensors 810.

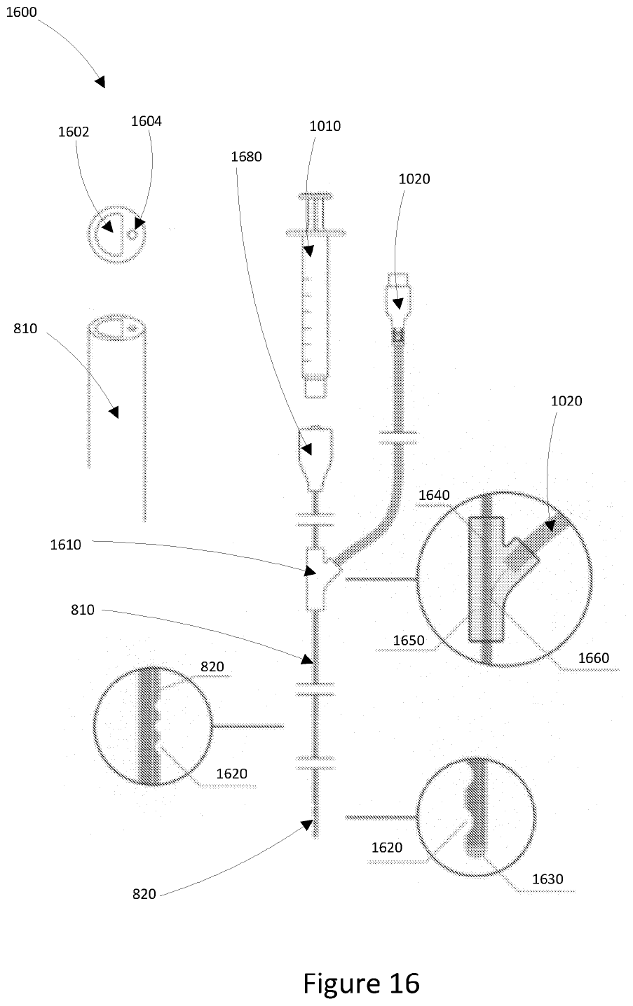

FIGS. 16 to 19 show some further embodiments of system 800 in further detail.

FIG. 16 shows a sub-system 1600 comprising device 810 with a junction 1610, syringe 1010 and data and power cable 1020. In sub-system 1600, device 810 comprises a multi-lumen or dual lumen, with a feeding lumen 1602 and a sensor lumen 1604. Feeding lumen 1602 may be several times larger than sensor lumen 1604. Sensor lumen 1604 may allow a fibre optic sensor 820 to be positioned within it, with perforations 1620 allowing for sensor 820 to be exposed to allow for pressure readings to be taken. The end of device 1630 is sealed.

In the illustrated embodiment, junction 1610 is a junction only with a separate taper or lock connector 1680 being connected to junction 1610 to allow a compatible syringe 1010 to be used to deliver substances through lumen 1602. Connector 1680 may be of any enteral feeding connector type. According to some embodiments, connector 1680 may be a Luer connector adapted to be used with a Luer fitting syringe. According to some embodiments, connector 1680 may be an ENFit connector adapted to be used with an ENFit syringe. Junction 1610 also allows for a data and power connection cable 1020 to be connected to sensors 820. Cable 1020 may comprise a PVC jacket according to some embodiments. Junction 1610 may comprise a seal 1640 at the top of optic fibre sensor 820, and an over-mould 1650. Junction 1610 may further comprise a perforation 1660 to allow cable 1020 to access sensors 820.

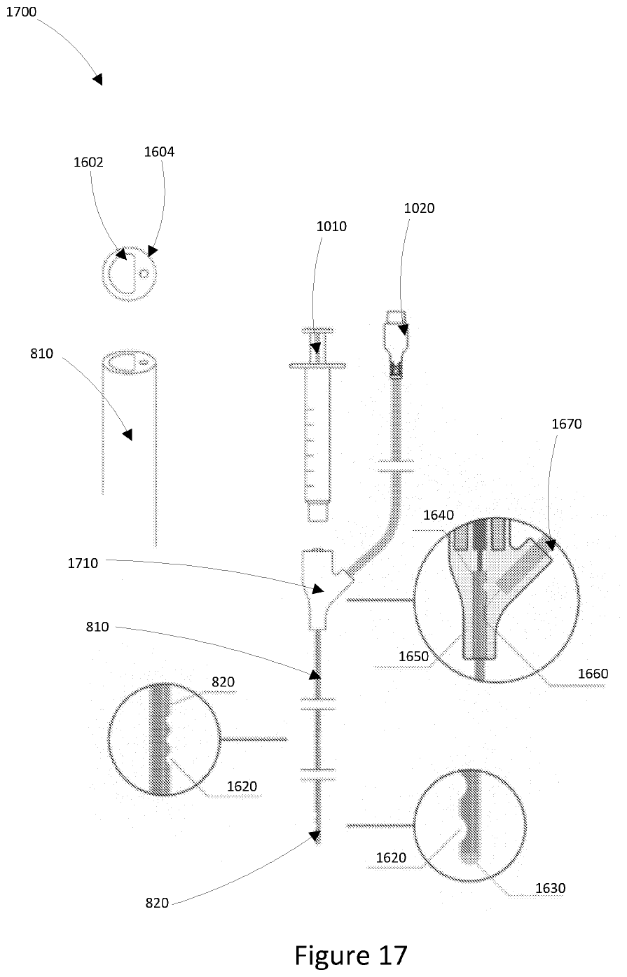

FIG. 17 shows an alternative sub-system 1700 comprising device 810 with a junction 1710, syringe 1010 and data and power cable 1020. Sub-system 1700 is similar to sub-system 1600, except that junction 1710 replaces junction 1610 and connector 1680. Instead, junction 1710 is a combined junction and taper or lock connector, allowing a compatible syringe 1010 to be used to deliver substances through lumen 1602. Junction 1710 may comprise any enteral feeding connector type. According to some embodiments, junction 1710 may comprise a Luer connector adapted to be used with a Luer fitting syringe. According to some embodiments, junction 1710 may comprise an ENFit connector adapted to be used with an ENFit syringe.

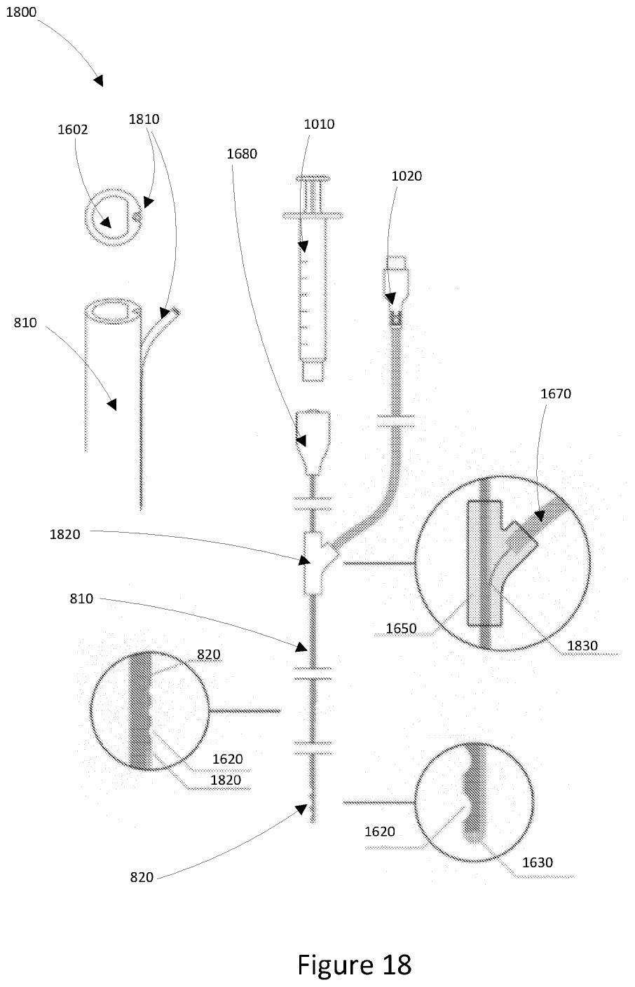

FIG. 18 shows a further alternative sub-system 1800 comprising device 810 with a junction 1820, syringe 1010 and data and power cable 1020. Sub-system 1700 is similar to sub-system 1600, except that junction 1820 replaces junction 1610, and device 810 of sub-system 1800 comprises two lumens 1602 and 1810 bonded with one or more optic fibre sensors. Junction 1820 is similar to junction 1610, except that instead of perforation 1660, junction 1820 has a split 1830 where the optic fibre sensor 820 splits out of lumen 1810 and away from device 810. Due to the split 1830, seal 1640 is not required.

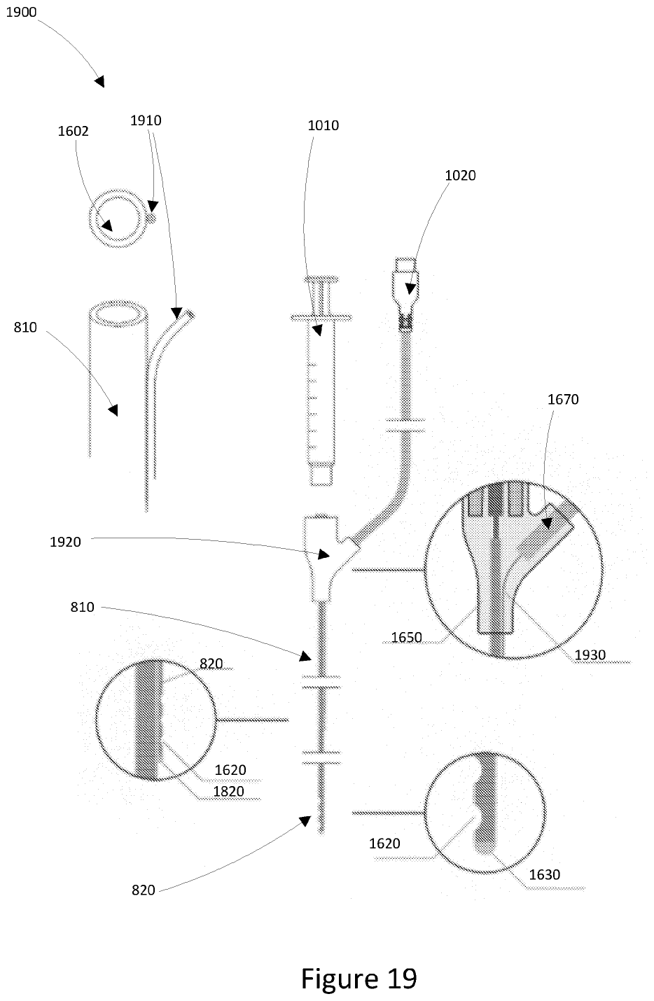

FIG. 19 shows another further alternative sub-system 1900 comprising device 810 with a junction 1920, syringe 1010 and data and power cable 1020. Sub-system 1900 is similar to sub-system 1700, except that junction 1820 replaces junction 1710, and device 810 of sub-system 1900 comprises a multi-lumen co-extruded with optic fibre sensors 820. The co-extruded multi-lumen comprises feeding lumen 1602 and sensor lumen 1910. Junction 1920 is similar to junction 1710, except that instead of perforation 1660, junction 1920 has a split 1930 where the optic fibre sensor 820 splits out of lumen 1910 and away from device 810. Due to the split 1930, seal 1640 is not required.

System 800 may be suitable for use in patients who require intermittent or continuous tube feeding via the naso/orogastric route and the simultaneous monitoring of airway pressure during respiratory support. This may include neonatal, infant and paediatric patients exhibiting Respiratory Distress Syndrome (RDS), Chronic Lung disease, apnea of prematurity, pneumonia, myopathy, muscle fatigue, impending of respiratory muscles, ventilator management, weaning, good respiratory drive but still requiring minimal respiratory support, and for lung collapse prevention. This may also include adult patients for acute lung injury, neuromuscular disorders and ventilator weaning.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A device for monitoring air pressure in the body of a patient, the device comprising:
a tube comprising a feeding lumen;
a sensor lumen positioned parallel to the feeding lumen;
at least one sensor; and
at least one perforation positioned to expose the at least one sensor to an air pressure within the body of a patient when the device is positioned at least partially within an airway of the patient;
wherein the at least one sensor is configured to generate data related to the pressure within the airway to which the sensor has been exposed.

2. The device of claim 1, wherein the tube comprises the sensor lumen and the at least one sensor does not protrude from an exterior of tube.

3. The device of claim 1, further comprising a sensor conduit coupled along the length of the tube, wherein the sensor conduit comprises the sensor lumen.

4. The device of claim 1, wherein the air pressure is at least one of laryngopharyngeal pressure, lower oesophageal sphincter pressure, lower oesophageal pressure and lung pressure.

5. The device of claim 1, further comprising a connector portion to allow the tube to be fluidly coupled to a feeding line and to allow the at least one sensor to be electrically coupled to a processing unit.

6. The device of claim 1, wherein the tube is at least one of a nasogastric or orogastric tube.

7. The device of claim 1, wherein the tube acts as an enteral feeding tube.

8. The device of claim 1, further comprising at least one positioning marker to assist in placing the device into at least one of the laryngopharyngeal, lower oesophageal sphincter and lower oesophageal region of the patient.

9. The device of claim 1, wherein the sensor is an optic fibre pressure sensor.

10. (canceled)

11. The device of claim 1, wherein the at least one sensor comprises at least two sensors positioned along a length of the tube.

12. (canceled)

13. The device of claim 1, wherein the at least one sensor comprises at least two sensors positioned around a circumference of the tube.

14. The device of claim 1, further comprising a light source to shine light through an anterior section of the patient's throat to assist in positioning of the device at least partially in the airway of the patient.

15. The device of claim 1, wherein monitoring air pressure in the body of a patient comprises monitoring air pressure within an upper digestive tract of the patient.

16. A system for monitoring air pressure in the body of a patient, the system comprising:
the device of claim 1; and
a processor unit configured to receive sensor data generated by the at least one sensor.

17. (canceled)

18. The system of claim 16, wherein the processing device is configured to determine at least one of a pressure administered by the device, a pressure being delivered to the airway, an indication of whether the pressure being delivered is outside a predetermined limit, an indication of whether an error exists in the device, and at least one parameter relating to airflow dynamics in the airway.

19. The system of claim 16, wherein the system is configured to detect airflow variations and to analyse airflow dynamics.

20. The system of claim 16, wherein the system is configured to detect gastroesophageal reflux.

21. The system of claim 16, wherein the system allows for real-time monitoring.

22. The system of claim 16, wherein the system is configured to determine an invalid sensor reading.

23. The system of claim 16, wherein the processor unit is configured to determine at least one of a respiratory rate and a heart rate based on the sensor data.
